(19) United States
(12) Patent Application Publication
(10) Pub. No.: US 2008/0300518 A1
(43) Pub. Date: Dec. 4, 2008

(54) SYSTEM, METHOD, AND APPARATUS FOR ASSISTING A RESCUER IN RESUSCITATION

(76) Inventor: C. J. Bowes, Fullerton, CA (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 12/131,376

(22) Filed: Jun. 2, 2008

Related U.S. Application Data
(60) Provisional application No. 60/941,376, filed on Jun. 1, 2007.

(51) Int. Cl. A61H 31/00; A61B 19/00; A61B 17/00

(52) U.S. Cl. 601/41; 128/898; 606/201

(57) ABSTRACT

A device, method and system are disclosed for assisting a rescuer attempting to resuscitate a patient in need of resuscitation. A CPR assist device is provided that includes a first indication of a first surface of the CPR assist device to place in contact with a patient. The CPR assist device further includes a second indication of a second surface of the CPR assist device to place in contact with a patient. In one embodiment, the CPR assist device is adapted to provide indicia to a rescuer that provide instructions to assist the rescuer to resuscitate the patient. In another embodiment, the CPR assist device is adapted to initiate or adjust instructions and/or therapy based on whether the first surface or the second surface of the CPR assist device is placed in contact with the chest region of a patient in need of resuscitation.

### RELATED APPLICATION

This application claims the benefit of U.S. Provisional Application No. 60/941,376, entitled SYSTEM, METHOD, AND APPARATUS FOR ASSISTING A RESCUER IN RESUSCITATION, filed Jun. 1, 2007, and which is hereby incorporated by reference in its entirety.

### TECHNICAL FIELD

The present invention relates to methods and devices useful for assisting in resuscitating a patient. More particularly, the present invention relates to a device adapted to be placed on the body of a patient to provide instruction and/or indicators to assist a rescuer when resuscitating the patient.

### BACKGROUND OF THE INVENTION

Many techniques exist for resuscitating a patient suffering from a life threatening condition. Patient conditions that may require resuscitation include, but are not limited to, cardiac arrest, bradycardia, tachycardia, ventricular fibrillation and respiratory arrest. One example of a technique for resuscitating a patient is performing CardioPulmonary Resuscitation (CPR) and/or mouth-to-mouth resuscitation.

CPR is a technique used by a rescuer in an emergency situation to get oxygen into a patients blood when the patient's heart has stopped beating and/or they are not breathing spontaneously. When performing CPR the rescuer creates blood circulation in the patient's body by periodically compressing the patient's chest. Mouth-to-mouth resuscitation is a technique used by a rescuer to initiate or assist a patient's breathing when the patient's heart has stopped beating and/or they are not breathing spontaneously. While performing mouth-to-mouth resuscitation the rescuer initiates or assists a patient's breathing-by-breathing into the mouth of the patient.

Chest compressions produce blood circulation as the result of a generalized increase in intrathoracic pressure and/or direct compression of the heart. The American Heart Association (“AHA”) guidelines state, “[b]lood circulated to the lungs by chest compressions will likely receive enough oxygen to maintain life when the compressions are accompanied by properly performed rescue breathing.” A patient can be kept alive using CPR provided the rescuer(s) are able to continue delivering properly performed chest compressions and rescue breaths.

The AHA recommends many different CPR techniques, depending on the patient and the patient's condition. (American Heart Association 2005 Guidelines for CPR and ECC). The AHA recommends that a rescuer use two hands for compressing an adult patients chest. For children, the AHA recommends using the heel of one hand for a smaller child or two hands for larger children. For infants, the AHA recommends two fingers for compressing a patient's chest when one rescuer is present, and two thumbs for compressing a patient's chest when two rescuers are present.

In 2005, the AHA simplified its recommendations to those stated above in order make CPR easier for laypersons to learn, remember, and perform. (American Heart Association 2005 Guidelines for CPR and ECC: Part 3, Overview of CPR). In making these simplifications, the AHA necessarily had to reduce the effectiveness of the different CPR techniques.

For this reason, there is a need for a practical device that provides a rescuer with information that indicates to the rescuer which of the different CPR techniques to use on a patient who has one or more particular attributes or one or more particular conditions. A device of this type will provide rescuers with coaching that will enable them to resuscitate a patient consistently and efficaciously according to the one or more particular attributes and/or one or more particular conditions. Because this device would be intended both for use by laypersons and medical professionals, there is further a need in the art for a practical device which will communicate with a large variety of potential rescuers.

Other devices exist to assist rescuers when resuscitating a patient. Examples of such devices include devices to monitor cardiac compression being applied to a patient. A user can apply force to these devices to provide cardiac compressions to the patient. These devices may also provide indications to assist a rescuer in performing CPR.

While existing devices can be used to assist a rescuer in providing CPR, the aforementioned devices do not provide the ability to select between different resuscitation techniques for a particular patient with one or more attributes or conditions, or a particular rescuer with one or more attributes or conditions.

### SUMMARY OF THE INVENTION

The present invention provides a device, method and system for assisting a rescuer in resuscitating a patient. In one embodiment, the device is an object sized and shaped such that it can be applied to the chest region of the patient to assist a rescuer in performing CPR. In various embodiments, the object includes a first indication that a first surface of the object is to be placed in contact with a patient. In another embodiment, the object further includes a second indication that a second surface of the object is to be placed in contact with a patient. In yet another embodiment, the object includes indicators located on the object to convey instructions indicating the use of the object when attempting to resuscitate a patient. In various alternative embodiments, the object includes one or more devices, wherein the one or more devices are adapted to convey instructions indicating the use of the object when attempting to resuscitate a patient.

According to one aspect, a system is provided for assisting a rescuer in resuscitating a patient. In an embodiment, the system includes a device referred to as a CPR assist device or CPR puck. In one embodiment, the CPR assist device is communicatively coupled to one or more external devices. In various embodiments, the one or more external devices are adapted to assist a rescuer in resuscitating a patient. In another embodiment, the one or more external devices are adapted to communicate instructions to a rescuer attempting to resuscitate a patient.

In various embodiments, a method of using a CPR assist device to resuscitate a patient is provided. In one embodiment, the method includes the step of a rescuer determining which surface of the CPR assist device to place in contact with the patient. This determination may be based on at least one first indication of at least one portion of a first surface of the CPR assist device to place in contact with a chest region of a patient and at least one second indication of at least one portion of a second surface of the CPR assist device to place in contact with the chest region of a patient. In another embodiment, the rescuer places either the first surface or the second surface of the CPR assist device in contact with the chest region of the patient, based on the determining step. In yet another embodiment, the method includes the step of the rescuer receiving instructions, wherein the instructions are based at least in part on whether the first surface or the second surface is placed in contact with the chest region of the patient.

According to one aspect of the present disclosure, a method of assisting a rescuer in resuscitating a patient using a CPR puck is provided. In various embodiments, the method includes the step of providing a CPR puck to the rescuer. In one embodiment, the CPR puck is sized and shaped to be disposed upon a chest region of a patient. In another embodiment, the method includes the step of providing to the rescuer a first indication that a first surface of the CPR puck is to be disposed upon the chest region of the patient. In yet another embodiment, the method includes the step of providing to the rescuer a second indication that a second surface of the CPR puck is to be disposed upon the chest region of a patient. In various embodiments, the method After includes the step of providing instructions to a rescuer for resuscitating a patient, wherein the instructions are based at least in part on whether the first surface or the second surface is disposed upon the chest region of the patient.

### BRIEF DESCRIPTION OF THE DRAWINGS

These, as well as other objects and advantages of this invention, will be more completely understood and appreciated by referring to the following more detailed description of the presently preferred exemplary embodiments of the invention in conjunction with the accompanying drawings, of which:

FIG. 1 illustrates generally the use of CPR to resuscitate a patient.

FIG. 2a illustrates generally a the recommended hand arrangement for performing CPR on an adult

FIG. 2b illustrates generally the recommended hand arrangement for performing CPR on a child.

FIG. 2c illustrates generally the recommended hand arrangement for performing CPR on an infant.

FIG. 3a illustrates generally a first surface of one embodiment of a CPR assist device.

FIG. 3b illustrates generally a second surface of one embodiment of a CPR assist device.

FIG. 3c illustrates generally a side view of one embodiment of a CPR assist device.

FIG. 3d illustrates generally a cross-sectional side view of an alternative embodiment of a CPR assist device.

FIG. 4 illustrates generally various examples of devices a CPR assist device may include.

FIG. 5 illustrates generally various examples of sensors a CPR assist device may include.

FIGS. 6a and FIG. 6b illustrates generally one embodiment of a CPR assist device

FIGS. 7a and FIG. 7b illustrate generally one embodiment of a CPR assist device.

FIGS. 8a and 8b illustrate generally one embodiment of a CPR assist device.

FIGS. 9a and 9b illustrate generally one embodiment of a CPR assist device.

FIGS. 10a-10b illustrate generally a rescuer using a CPR assist device to resuscitate a patient.

FIG. 11 illustrates generally a flow chart diagram of assisting a rescuer in performing patient resuscitation by providing a CPR assist device.

FIG. 12 illustrates generally examples of instructions provided by the CPR assist device.

FIG. 13 illustrates generally a system including a CPR assist device.

### DETAILED DESCRIPTION OF THE DRAWINGS

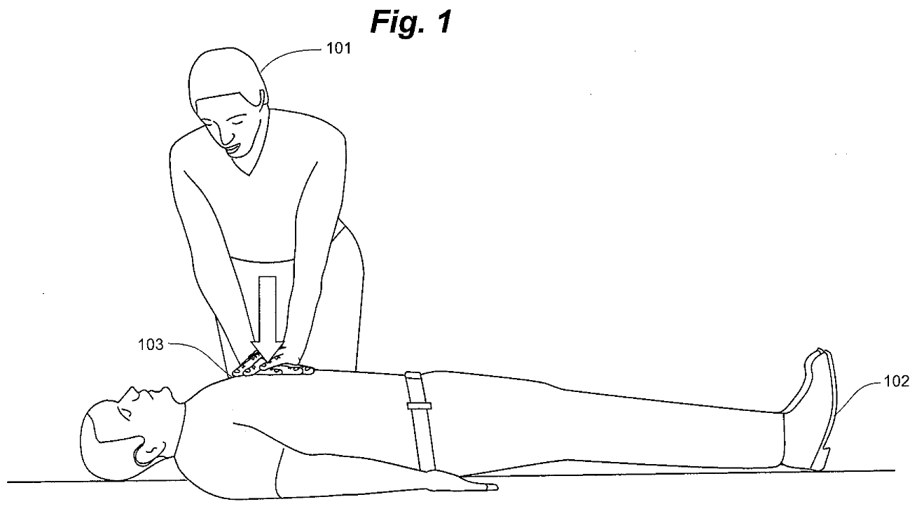

FIG. 1 illustrates generally a rescuer 101 performing CPR on a patient 102. Rescuer 101 performs CPR by placing one or more hands on the chest region of patient 102 and compressing the chest 103 of patient 102. Chest compressions may be performed at various different intervals and compression depths depending on the patients condition and the patients attributes. Rescuer 101 may also perform Mouth-to-mouth resuscitation along with performing CPR. Patient conditions that may require resuscitation include, but are not limited to, cardiac arrest, bradycardia, tachycardia, ventricular fibrillation and respiratory arrest. Various examples of a patient's attributes include the patients age, the patient's size, and the patient's sex. Various examples of a rescuer's condition or attributes include the language understood by the rescuer, the rescuer's size, the rescuer's age, or the rescuer's familiarity of resuscitation procedures.

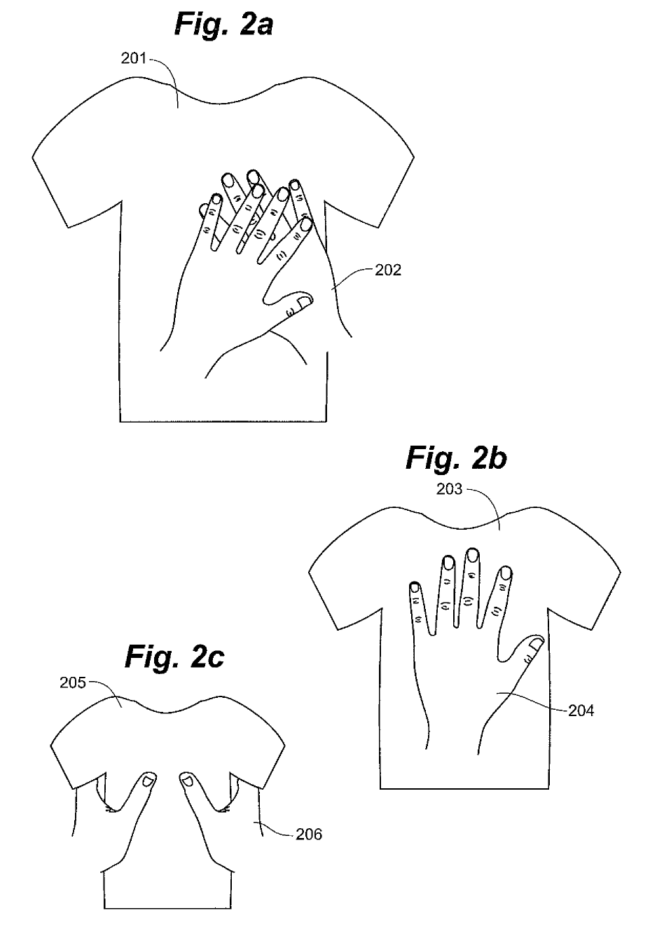

FIGS. 2a to 2c illustrate generally examples of different CPR techniques for resuscitating patients based on an attribute of a patient. FIG. 2a illustrates the method of hand placement 202 for a rescuer on the chest 201 of a patient when performing CPR on an adult patient. In this example, a rescuer of an adult patient may place at least one hand upon the chest 201 of the adult patient. In another example, a rescuer of an adult patient may place both hands on the chest 201 of the adult patient. In the example of FIG. 2a, the rescuer places the palm of one hand upon the chest 201 of a patient, places the palm of his/her other hand on the first hand, and uses both hands to compress the chest of the patient.

FIG. 2b illustrates the method of hand placement 204 of a rescuer on the chest 203 of a patient when performing CPR on a child patient. The AHA defines a child as one of age greater than 1 year who has not yet reached puberty. In this example, a rescuer of a child patient may place one hand on the chest 203 of the patient, or two hands on the chest 204 of the patient. The rescuer may place the palms of both hands on the chest 203 of the patient, the palm of one hand with the palm of his/her other hand upon the first hand, or one hand alone upon the chest 203 of a patient. Once the rescuer has placed his hands upon the chest 203 of a patient, the rescuer then proceeds to apply chest compression to resuscitate the patient.

FIG. 2c illustrates the method of hand placement 206 of a rescuer's hands upon the chest 205 of a patient when performing CPR on an infant patient, The AHA defines an infant as one who's age is less than or equal to one year. In this example, the hand placement of a rescuer depends on whether the rescuer is a sole rescuer or whether a second rescuer is present. If the rescuer is a sole rescuer, the AHA recommends that the rescuer place a finger from each hand upon the chest 205 of the patient. If a second rescuer is present, the AHA recommends that the rescuer place two thumbs upon the chest 205 of the patient. Once the rescuer has placed his thumbs or fingers upon the chest 205 of the patient, the rescuer then proceeds to apply chest compression to resuscitate the patient.

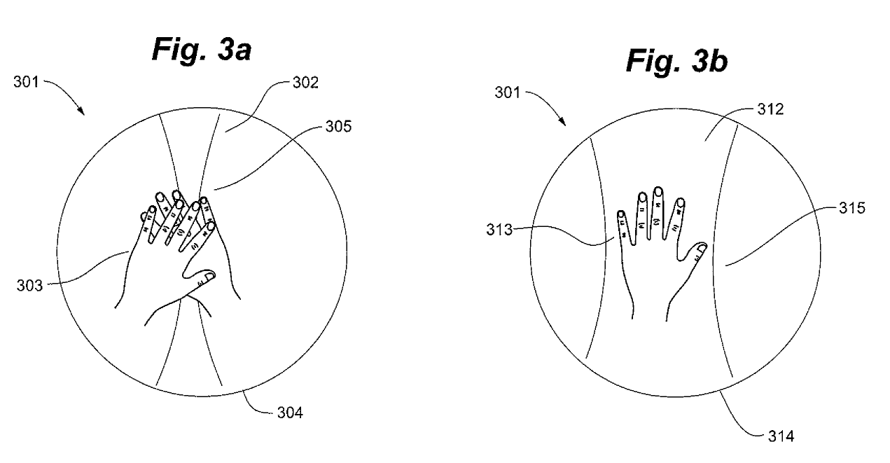

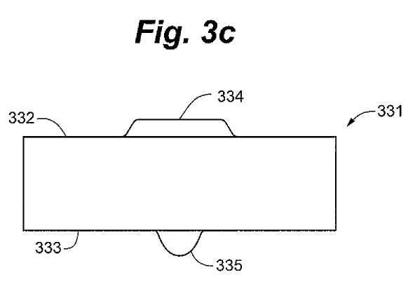

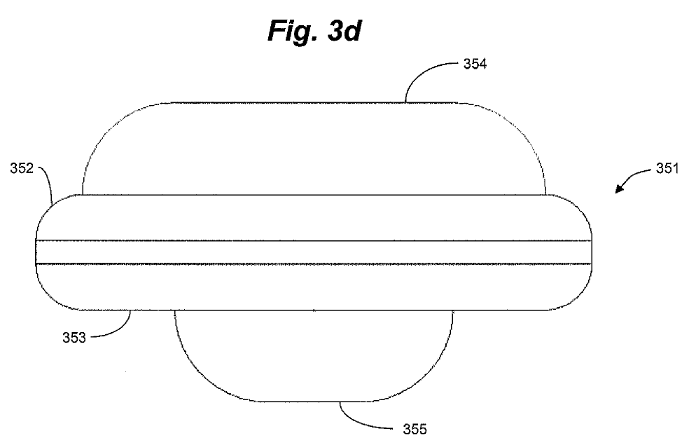

FIG. 3 illustrates generally one embodiment of a CPR puck 301 according to the present disclosure. The term “puck” is used herein for purposes of illustration, not for purposes of limitation. The term “puck” as used herein is intended to encompass any object sized and shaped with at least one surface to be placed upon the chest region of a patient in need of resuscitation where the dimensions surface area are generally greater than an average depth of the object. A CPR “puck” as used herein, refers to a CPR assist device, CPR chest compression sensor, or CPR coaching device and may alternatively be referred to by such terms at times throughout this disclosure. FIG. 3a illustrates a first surface 304 of a CPR assist device or CPR puck 301, while FIG. 3b illustrates a second surface 314 of a CPR assist device or CPR puck 301. In this embodiment, CPR puck 301 is an object. In various embodiments, the object is sized and shaped to be disposed upon a chest region of a patient. In an embodiment, CPR puck 301 may be composed of a soft compressible material. In another embodiment, CPR puck 301 may be composed of a hard material. In various embodiments, CPR puck 301 may be composed of any material capable of being sized and shaped to be disposed upon a chest region of a patient.

FIGS. 3a and 3b further illustrate an embodiment wherein first surface 304 and second surface 314 are adapted to substantially conform to the chest region of a patient. According to this embodiment, when a rescuer is attempting to resuscitate an adult patient, second surface 314 is placed in contact with the adult patient. Further, in accordance with this embodiment, second surface 314 is adapted to substantially conform to the chest region of an adult patient. When a rescuer is attempting to resuscitate a child patient, first surface 304 is placed in contact with the child patient. According to this embodiment, first surface 304 is adapted to substantially conform to the chest region of a child patient. Because the chest region of an adult patient is larger than the chest region of a child patient, the portion of the surface adapted to conform to the chest region of an adult patient 315 is wider than the portion of the surface adapted to conform to the chest region of a child patient 305.

FIG. 3c illustrates generally a side view of an embodiment of a CPR puck 331. According to this embodiment, CPR puck 331 includes a first surface 332 adapted to conform to the chest region of a first group of patients and a second surface 333 adapted to conform to the chest region of a second group of patients. According to this embodiment, the first group of patients is larger than the second group of patients. Therefore, the portion 334 adapted to substantially conform to the chest region of a first group of patients is larger than the portion 335 adapted to substantially conform to the chest region of a second group of patients.

FIG. 3d illustrates generally an alternative side cross-sectional view of an alternative embodiment of a CPR puck 351. Such an embodiment may have various geometries and is not intended to be limited to the shapes and contours disclosed in the figures. In this embodiment, the CPR puck 351 has a first surface 352 adapted to conform to the chest region of a first group of patients and a second surface 353 adapted to conform to the chest region of a second group of patients. According to this embodiment, the first group of patients is larger than the second group of patients. Therefore, the portion 354 adapted to substantially conform to the chest region of a first group of patients is larger than the portion 355 adapted to substantially conform to the chest region of a second group of patients.

According to one aspect, the object includes at least two surfaces 304 and 314. The examples of FIGS. 3a and 3b illustrate a circular shape to the at least two surfaces 304 and 314, however, according to various examples, surfaces 304 and 314 can be in the shape of a circle, an oval, a square, a rectangle, or any other shape. In various embodiments, CPR puck 301 includes a first indicator 302 and a second indicator 312. In an embodiment, first indicator 302 indicates that first surface 304 of CPR puck 301 is to be placed in contact with the patient. In an embodiment, second indicator 312 indicates that second surface 314 of the CPR puck 301 is to be placed in contact with the patient. In the embodiment illustrated in FIGS. 3a and 3b, first indicator 302 indicates “Adult”. In this embodiment, first indicator 302 indicates to a rescuer that second surface 314 of the CPR puck 301 should be placed in contact with the chest region of a patient if the patient is an adult. In the embodiment illustrated in FIG. 3b, the second indicator 312 indicates “Child”. In this embodiment, the second indicator 312 indicates to a rescuer that the first surface 304 of the CPR puck 301 should be placed in contact with the chest region of a patient when that patient is a child.

In various embodiments, CPR puck 301 may include indicators thereon, to convey instructions as to the use of the CPR puck 301 and the AHA recommended technique for resuscitating a particular patient. In one embodiment, the indicators may include one or more indications of the AHA recommended hand placement for performing CPR. The embodiment shown in FIG. 3a and FIG. 3b further illustrates hand positioning indicators 303, 313. In this embodiment, indicators on first surface 304 include a hand positioning indicator 303. Hand positioning indicator 303 may be located in the center region of CPR puck 301. Hand positioning indicator 303 indicates to a rescuer that when attempting to resuscitate an adult both hands should be used as described herein. In an embodiment indicator on second surface 314 includes a hand positioning indicator 313 that may be located in the center region of the CPR puck 301. Hand positioning indicator 313 indicates to a rescuer that when attempting to resuscitate a child a single hand should be used as described herein.

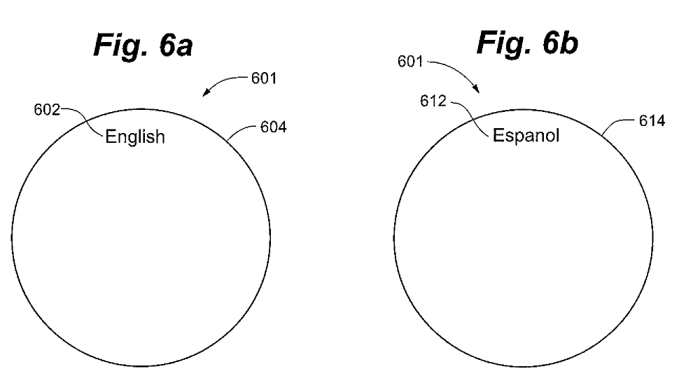

FIG. 6a and FIG. 6b illustrate generally one embodiment of a CPR puck 601. FIG. 6a illustrates a first surface 604 of CPR puck 601, while FIG. 6b illustrates a second surface 614 of a CPR puck 601. In various embodiments, CPR puck 601 may include at least two surfaces 604 and 614. In an embodiment, CPR puck 601 may include a first indicator 602 and a second indicator 612. In one embodiment, first indicator 602 indicates that second surface of the object 614 is to be placed in contact with the patient. In an embodiment, second indicator 612 indicates that the first surface of the object 604 is to be placed in contact with the patient.

According to one aspect, first indicator 602 indicates to a rescuer that CPR puck 601 will communicate in a first language. In another embodiment, a second indicator 612 indicates that the CPR puck 601 will communicate in a second language. According to these embodiments, CPR puck 601 may be adapted to communicate in a first language if second surface 614 is placed in contact with a patient. Further, according to these embodiments, CPR puck 601 may be adapted to communicate in the second language if first surface 604 is placed in contact with the patient. In alternate embodiments, CPR puck 601 may be adapted to communicate in a first language if first surface 604 is placed in contact with a patient. In another alternative embodiment, CPR puck 601 may be adapted to communicate in a second language if second surface 614 is placed in contact with a patient.

In the exemplary embodiment illustrated in FIGS. 6a and 6b, first indicator 602 indicates “English”. In this embodiment, first indicator 602 indicates to a rescuer that a second surface 614 of CPR puck 601 should be placed in contact with the chest region of a patient if the rescuer understands the English language. In the embodiment illustrated in FIG. 6b, second indicator 612 indicates “Espanol”. In this embodiment, second indicator 612 indicates to a rescuer that first surface 604 of CPR puck 601 should be placed in contact with the chest region of a patient if the rescuer understands the Spanish language. In various embodiments, as discussed herein, CPR puck 601 may be adapted to communicate instructions to one or more rescuers who understand one or more different languages.

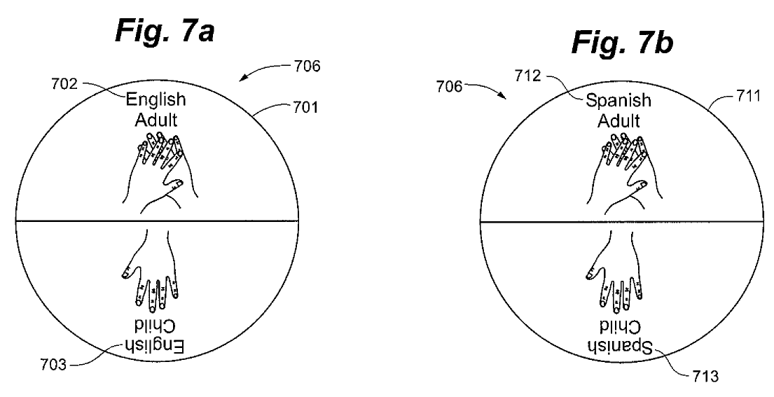

FIGS. 7a and 7b illustrate generally an alternative embodiment of a CPR puck 706. FIG. 7a illustrates generally a first surface 701 of CPR puck 706. FIG. 7b illustrates a second surface 711 of CPR puck 706. According to the embodiment illustrated in FIG. 7a, first surface 701 includes both a first indicator 702 and a second indicator 703. According to this embodiment, both first indicator 702 and second indicator 703 of first surface 701 may indicate “English.” This indication informs a rescuer that CPR puck 706 is adapted to communicate in the English language. According to this embodiment, first indicator 702 further indicates to a rescuer “Adult.” Further, according to this embodiment, if a rescuer is attempting to resuscitate an Adult patient, the rescuer orients CPR puck 706 on the chest region of a patient such that first indicator 702 is closest to the head of the patient. According to this embodiment, second indicator 703 further indicates to a rescuer “Child.” If a rescuer is attempting to resuscitate a Child patient, the rescuer orients CPR puck 706 on the chest region of a patient such that second indicator 703 is closest to the head of the patient.

In one embodiment, second surface 711 may include both a first indicator 712 and a second indicator 713. According to this embodiment, both first indicator 712 and second indicator 713 of second surface 711 may indicate “Espanol.” This indication informs a rescuer that CPR puck 706 is adapted to communicate in the Spanish language. According to this embodiment, first indicator 712 further indicates to a rescuer “Adult.” According to this embodiment, if a rescuer is attempting to resuscitate an Adult patient, the rescuer orients CPR puck 706 on the chest region of a patient such that first indicator 712 is closest to the head of the patient. According to this embodiment, second indicator 713 further indicates to a rescuer “Child.” If a rescuer is attempting to resuscitate a Child patient, the rescuer orients CPR puck 706 on the chest region of a patient such that second indicator 713 is closest to the head of the patient.

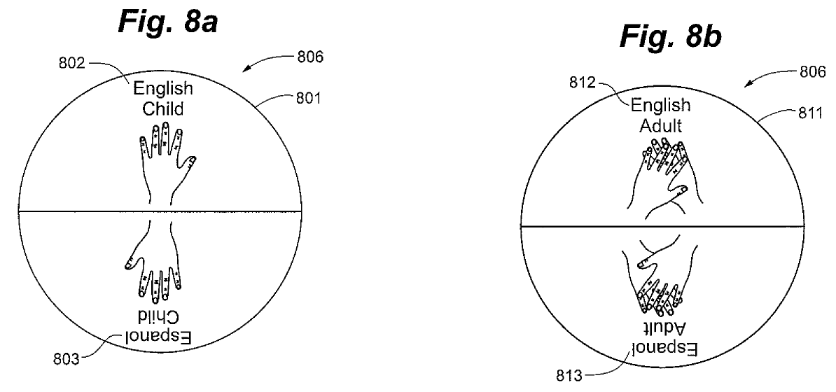

FIGS. 8a and 8b illustrate generally another embodiment of a CPR puck 806. FIG. 8a illustrates generally a first surface 801 of CPR puck 806. FIG. 8b illustrates a second surface 811 of CPR puck 806. In one embodiment, first surface 801 includes both a first indicator 802 and a second indicator 803. According to this embodiment, both first indicator 802 and second indicator 803 of first surface 801 indicate “Child.” This indication informs a rescuer that CPR puck 806 is intended for use on a patient who is a child. According to this embodiment, first indicator 802 further indicates to a rescuer “English.” If a rescuer understands the English language, the rescuer orients CPR puck 806 on the chest region of a patient such that first indicator 802 is closest to the head of the patient. According to this embodiment, second indicator 803 further indicates to a rescuer “Espanol.” If a rescuer understands the Spanish language, the rescuer orients CPR puck 806 on the chest region of a patient such that the second indicator 803 is closest to the head of the patient.

FIG. 8b illustrates generally a second surface 811 of CPR puck 806. In one embodiment, second surface 811 includes both a first indicator 812 and a second indicator 813. According to this embodiment, both first indicator 812 and second indicator 813 indicate “Adult.” These indications inform a rescuer that CPR puck 806 is intended for use on a patient who is an Adult. According to this embodiment, first indicator 812 further indicates to a rescuer “English.” If a rescuer understands the English language, the rescuer orients CPR puck 806 on the chest region of a patient such that first indicator 812 is closest to the head of the patient. According to this embodiment, the second indicator 813 further indicates to a rescuer “Espanol.” If a rescuer understands the Spanish language, the rescuer orients CPR puck 806 on the chest region of a patient such that the second indicator 813 is closest to the head of the patient.

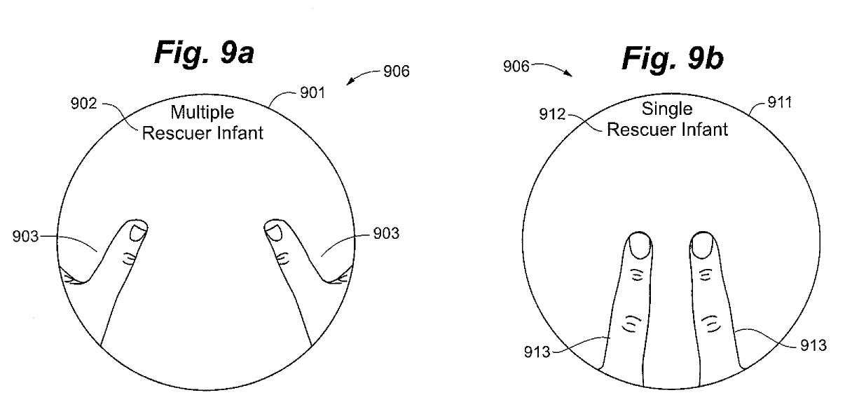

FIGS. 9a and 9b illustrate generally an alternative embodiment of a CPR puck. FIG. 9aillustrates generally a first surface 901 of CPR puck 906. FIG. 9b illustrates a second surface 911 of CPR puck 906. According to the AHA, when one rescuer is attempting to resuscitate an infant patient, the rescuer should place two fingers upon the chest region of a patient when attempting to compress the chest of the patient. Alternatively, the AHA further recommends that when more than one rescuer is attempting to resuscitate an infant patient, the rescuer who is performing chest compression should place two thumbs on the chest of the patient.

According to the embodiment of FIGS. 9a and 9b, first surface 901 includes the indicator “Multiple Rescuer Infant” 902. Indicator 902 informs one or more rescuers that CPR puck 906 is intended for use on an infant when more than one rescuer is attempting to resuscitate the infant. FIG. 9b illustrates generally a second surface 911 of CPR puck 906. In one embodiment second surface 911 includes the indicator “Single Rescuer Infant” 912. Indicator 912 informs a single rescuer that CPR puck 906 is intended for use on an infant when one rescuer is attempting to resuscitate the infant. First surface 901 includes a further indicator 903 that in the case of multiple rescuers, the rescuer attempting to compress the chest of an infant patient should place two thumbs on CPR puck 906. Second surface 911 includes indicator 913 indicating that a single rescuer of an infant patient should place two fingers on CPR puck 906 in order to compress the chest of the infant patient.

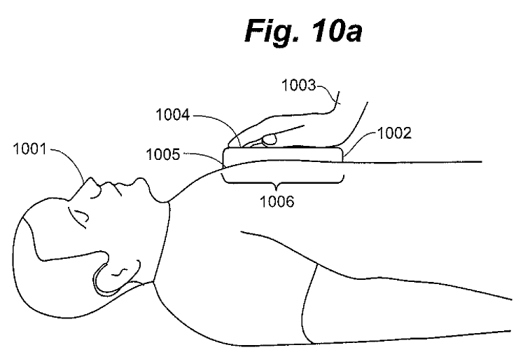

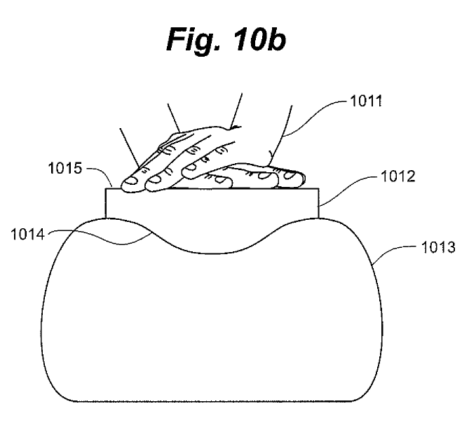

FIG. 10a illustrates generally an embodiment of a method of resuscitating a patient wherein a rescuer 1003 uses CPR puck 1002 to resuscitate a patient 1001. According to this embodiment the method includes the step of the rescuer determining, which of first surface 1004 or second surface 1005 to place in contact with the chest region 1006 of the patient 1001. In at least one embodiment, this step includes the rescuer determining which surface to place on the patient 1001 based on at least one first indicator on at least one portion of a first surface 1004 of CPR puck 1002, and at least one second indicator on at least one portion of a second surface 1005 of CPR puck 1002. The rescuer then places, based on the preceding determination, either the first surface 1004 or the second surface 1005 in contact with the chest region 1006 of the patient 1001. In the example illustrated in FIG. 10a, the rescuer has placed second surface 1005 in contact with chest region 1006 of patient 1001.

FIG. 10b illustrates generally an alternative side view of an embodiment of rescuer 1011 using CPR puck 1012 to resuscitate patient 1013. According to this example, as discussed herein, the rescuer determines which surface of CPR puck 1012 to place in contact with a patient. In this example, the rescuer 1011 placed a first surface 1014 of CPR puck 1012 in contact with patient 1013. According to this embodiment, CPR puck 1012 is adapted to conform to the chest region of the patient 1013. In this embodiment, the rescuer 1011 received instructions based on an indicator included on a surface 1015 of the CPR puck, and according to the indicator the rescuer 1011 placed both hands on CPR puck 1012 in order to perform chest compression on the patient 1013.

In various embodiments, the rescuer receives instructions based at least in part on the CPR puck 1002. In one embodiment, the instructions include indicators on either first surface 1004 or second surface 1005 of CPR puck 1002. In the example illustrated in FIG. 10a, the indicators are located on first surface 1004 of CPR puck 1002. In various examples, the rescuer may receive instructions from the indicators such as: hand placement, compression rate, compression depth, rate of mouth-to-mouth resuscitation, or any other instruction relevant to resuscitating a patient 1001. According to the embodiment illustrated in FIG. 10a, the rescuer then proceeds to attempt resuscitating the patient. Attempting to resuscitate the patient may include exerting force upon CPR puck 1002 in order to compress the chest of the patient.

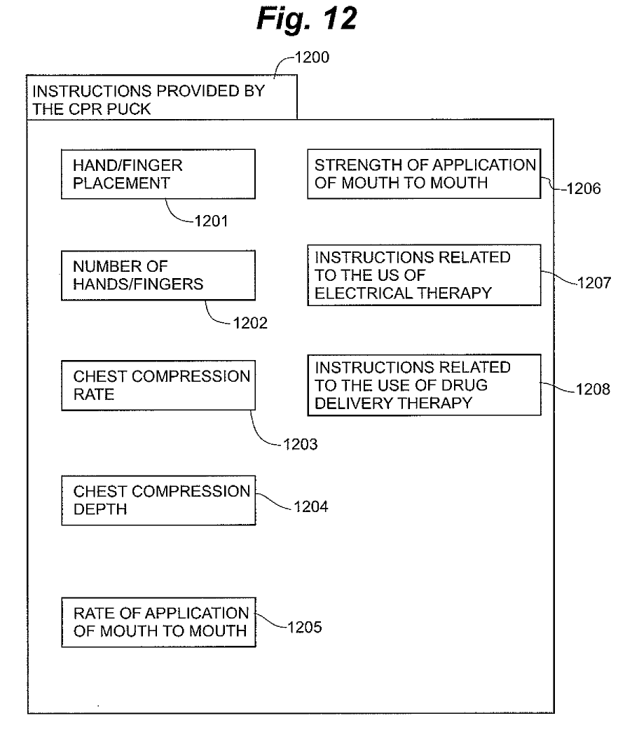

FIG. 12 illustrates generally embodiments of instructions 1200 provided to the rescuer. In various examples, provided for purposes of illustration, not limitation, the rescuer may receive instructions, based at least in part on whether first surface 304 or second surface 314 of CPR puck such as illustrated in FIGS. 3a and 3b is placed in contact with the patient. Instructions 1200 may include but are not limited to: hand/finger placement 1201, number of hand/fingers 1202, chest compression rate 1203, chest compression depth 1204, rate of application of mouth-to-mouth 1205, strength of application of mouth-to-mouth 1206, instructions related to the use of electrical therapy 1207, instructions related to the use of drug delivery therapy 1208, or any other instruction relevant to resuscitating a patient. In various examples, instructions 1200 take the form of indicators on one or more surfaces of CPR puck 301

According to one aspect of the present disclosure, a rescuer may receive audio or visual instructions. In an embodiment, CPR puck 301 is adapted to communicate audio or visual instructions. In one embodiment, CPR puck 301 is adapted to communicate visual instruction in the form of emitted light. In another embodiment, CPR puck 301 is adapted to communicate visual instructions through an illumination device. In one embodiment, such an illumination device is an LED (“light emitting diode”). In this example, one or more LED may communicate instructions through a series of illuminations that provide instructions to a rescuer. Such instructions may include, but are not limited to: when to compress the chest of a patient, or when to administer mouth-to-mouth resuscitation. In one embodiment, CPR puck 301 is adapted to communicate visual instructions through displayed text. According to this embodiment, CPR puck 301 may include a display device to provide a rescuer with text instructions.

In various embodiments, CPR puck 301 may be adapted to communicate instructions such as chest compression rate through sound. In one embodiment, CPR puck 301 may be adapted to communicate instructions through a series of beeps or other noises that indicate instructions to a rescuer. In another embodiment, CPR puck 301 may be adapted to synthesize or recreate a human voice. In yet another embodiment, CPR puck 301 may be adapted to communicate instructions to the rescuer in the form of verbal instructions. In various embodiments, CPR puck 301 may be adapted to communicate any combination of audio or visual instructions.

In an embodiment, CPR puck 301 may be adapted to provide instructions that are at least in part dependant on sensed measurements of patient specific parameters. In one embodiment, the CPR puck 301 may be adapted to provide instructions based on sensed measurements of hemodynamic status. In another embodiment, the CPR puck 301 may be adapted to provide instructions based on sensed measurements of a resuscitation attempt. In yet another embodiment, the CPR puck 301 may be adapted to provide instructions based on measurements received using any sensor, including sensors as discussed hereinbelow.

In various embodiments, the CPR puck 301 may be adapted to initiate the production of instructions and/or adjust the instructions based on one or more inputs provided by one or more rescuers attempting to resuscitate a patient. In an embodiment, the one or more inputs are received through any input device, including input devices as discussed further hereinbelow.

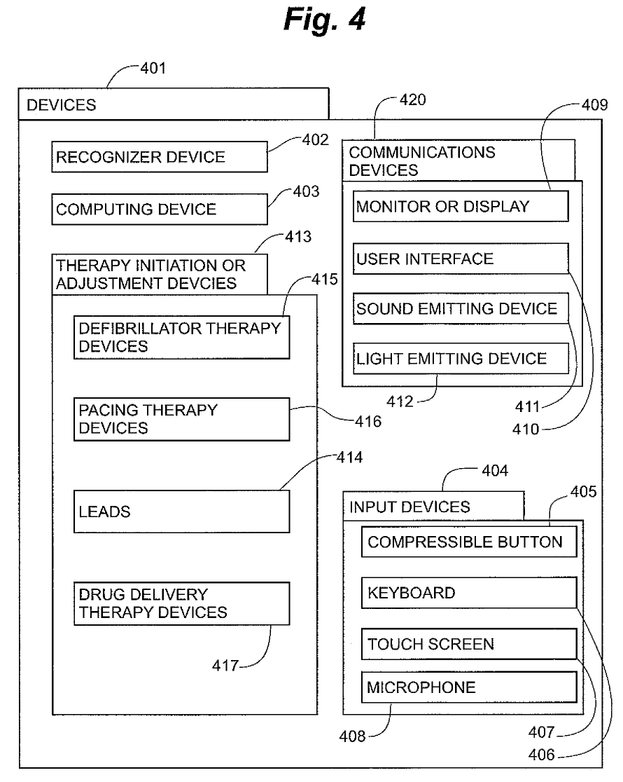

FIG. 4 illustrates generally various embodiments of devices that may be incorporated into the CPR puck 401. According to an embodiment the CPR puck 401 includes a recognizer device 402. The recognizer device 402 may be adapted to automatically determine what surface of CPR puck 401 is placed in contact with a patient. In one embodiment, the recognizer device 402 may be an accelerometer.

Accelerometers are well-known devices for measuring acceleration and gravity induced reaction forces. Various accelerometers may be readily incorporated into embodiments of this invention to detect the upward or downward facing direction of the CPR puck 301, including various tilt sensing accelerometers of a similar type to those commonly used in PDAs, cell phones, video games, etc.

A general teaching of such accelerometers is discussed in an Application Note by Kimberly Tuck, entitled “Tilt Sensing Using Linear Accelerometers”, Freescale Semiconductor, AN3461, 2007. Use and incorporation of accelerometers is well-known by persons of skill in the art. Disclosure of some possible commercially available accelerometer examples include Application Data Sheets for a number of devices sold by Analog Devices including iMems® Accelerometers ADXL320, ADXL330, ADIS 16209 as well as technical data sheets for the MMA7455L: XYZ Axis Accelerometer sold by Freescale Semiconductor, Inc. Each of these references is hereby incorporated by reference herein.

Coupling such an accelerometer with the CPR puck 301 enables an output providing automatic recognition of the orientation of the faces of the puck 301, wherein the downward directed face is placed in contact with the chest of the horizontally disposed patient. Accordingly, instructions or treatment is provided based on this orientation. Additional accelerometers may be used to ensure that appropriate treatment and instructions are provided if the patient is not in a fully horizontal position.

In addition to the use of accelerometers as a recognizer device 402, various other means are possible to automatically determine which surface of the CPR puck is in contact with a patient. These include pendulum sensors, gravity operated switches, optical sensors, temperature sensors, small resistive sensors, and sensors for detecting AED impedance stimulus signals or other signals provided by the system. For example, some embodiments may include use of a weighted switch to determine the directions of up and down with respect to the CPR puck. Alternatively, some embodiments may include use of a conductive fluid in a capsule to determine the directions of up and down with respect to the CPR puck, by having that conductive fluid touch electrical contacts based on the orientation of the puck. It is understood that a person having skill in the art could use and adapt such readily available sensors and other means to construct a device for automatically recognizing which surface of the CPR puck is in contact with a patient. Various modifications may be apparent to one of skill in the art upon reading this disclosure.

A recognizer device may also be desired to detect other types of puck orientation. In some embodiments, such as those shown in FIGS. 7a-b and 8a-b, recognition of the orientation of the puck 706 or 806 relative to specific features of the patient may be desired. More particularly, this relates to detecting whether the puck is in a first position or a second position that is rotationally related to the first position, where the device remains situated roughly in the same horizontal plane in both positions. One manner of detecting such a difference in orientation is through use of a Light-emitting diode (LED) and detector coupled to the puck 301. In such a device, the LED could be coupled to the puck 301 such that the light emitted would be unobstructed when the puck was oriented in one direction. For example, in the embodiment shown in FIG. 7a this may occur if indicator 702 were placed closest to the head of the patient. Alternatively, the light would be obstructed by a feature of the patient's body, such as the patient's chin, when the puck was rotated and thereby oriented in a second direction. For example, this may occur if the indicator 703 were placed closest to the head of the patient. Recognition of this interference by the detector accordingly provides automatic detection of the orientation of the puck relative to the head or other body feature of the patient. Instructions can thereby be provided based upon detection of this information. Alternatively, use of various other types of well-known sensors may be utilized as well.

In various embodiments, CPR puck 301 may include one or more computing devices 403. In an embodiment, the one or more computing devices 403 may be adapted to receive, process, and/or transmit information relevant to resuscitating a patient.

In various embodiments, the CPR puck 301 may be adapted to receive input from one or more rescuers. In an embodiment, CPR puck 301 may include an input device 404. In one embodiment, input device 404 may be a set of compressible buttons adapted to receive input 405. In an embodiment, input device 404 may be a keyboard 406. In another embodiment, input device 404 may be a touch-screen 407. In yet another embodiment, input device 404 may be a microphone 408. In a farther embodiment, microphone 408 may be communicatively coupled to a device adapted to perform voice recognition. In one embodiment, microphone 408 is communicatively coupled to a computing device 403 capable of performing voice recognition. In another embodiment, computing device 403 is adapted to recognize voice instructions provided by one or more rescuers attempting to resuscitate a patient.

In various embodiments, CPR puck 301 may include one or more communication devices 420. In one embodiment, communication device 420 may be one or more displays or monitors 409. In another embodiment displays or monitors 409 may be adapted to provide instructions to one or more rescuers attempting to resuscitate a patient. In yet another embodiment, displays or monitors 409 may be communicatively coupled to one or more computing devices 403. In various embodiments, CPR puck 301 may include one or more user interfaces 410. In an embodiment, computing devices 403 may be adapted to provide one or more user interfaces 410, using monitors or displays 409, to communicate with a rescuer. In various embodiments, user interfaces 410 may be adapted to receive input from and communicate with one or more rescuers.

In various embodiments, CPR puck 301 may include one or more sound emitting devices 411. Sound emitting devices 411 may include one or more speakers. In an embodiment, sound emitting devices 411 may be adapted to synthesize a human voice. In one embodiment, sound emitting device 411 may be adapted to provide audio instructions as discussed herein.

According to one aspect, CPR puck 301 may include one or more lights, such as light emitting devices (LEDs) 412. In one embodiment, light emitting devices 412 may include one or more LEDs. In an embodiment, the light emitting devices 412 are adapted to provide visual instructions to a rescuer as discussed herein.

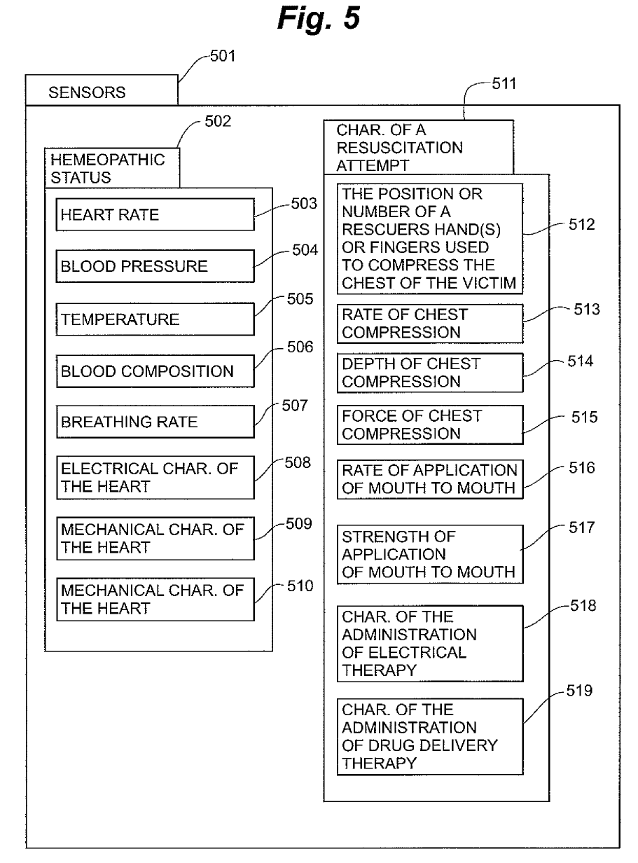

FIG. 5 illustrates generally various embodiments of CPR puck 301 includes one or more sensors 501. In various embodiments, CPR puck 301 may include one or more sensors adapted to sense parameters of a patient's health status. Examples of such parameters of health status 502, provided for purposes of illustration not limitation, include heart rate 503, blood pressure 504, temperature 505, blood composition 506, breathing rate 507, electrical characteristics of the heart 508, mechanical characteristics of the heart 509, and/or mechanical characteristics of the lungs 510. Other examples of sensors that may be utilized with CPR puck include: hemeopathic status sensors, blood pressure sensors, temperature sensors; blood composition sensors, breathing rate sensors, electrical heart characteristic sensors, mechanical heart characteristic sensors, and mechanical lung characteristic sensors.

In an embodiment, CPR puck 301 may include one or more sensors adapted to sense characteristics of a rescuer attempting to resuscitate a patient 511. Examples of such sensors, provided for purposes of illustration not limitation, include sensors adapted to determine: the position or number of a rescuers hand(s) or fingers used to compress the chest of the patient 512, the rate of chest compression 513, the depth of chest compression 514, the force of chest compression 515, the rate of application of mouth-to-mouth 516, the strength of application of mouth-to-mouth 517, characteristics of the administration of electrical therapy 518, and characteristics of the administration of drug delivery therapy 519, or any other instruction relevant to resuscitating a patient.

In various embodiments, CPR puck 301 may include one or more therapy devices 413 to initiate or adjust a therapy to assist in resuscitating a patient. In an embodiment, therapy devices 413 may be electrical therapy devices. In another embodiment, CPR puck 301 may include one or more leads 414. In an embodiment, such electrical therapy devices may include one or more defibrillation therapy devices 415, one or more pacing therapy devices 416, or both. Examples of pacing therapy devices 416 include, but are not limited to, a tachycardia therapy device and/or a bradycardia therapy device. In one embodiment, CPR puck 301 may provide instructions to a rescuer instructing the rescuer to initiate or adjust the electrical therapy. In another embodiment, the CPR puck is adapted to automatically or semi-automatically perform electrical therapy when in contact with the chest region of a patient.

In an embodiment, CPR puck 301 may include one or more medicant delivery therapy devices 417. In various embodiments, medicant delivery therapy devices 417 may deliver drugs, bioactive agents or any other suitable medicant subcutaneously, percutaneously, transdermally or by any other suitable method of delivery. In an embodiment, CPR puck 301 may provide instructions to a rescuer instructing the rescuer to initiate or adjust the medicant delivery therapy. In other embodiments, CPR puck 301 may be adapted to automatically or semi-automatically initiate or adjust medicant delivery therapy when in contact with the chest region of a patient.

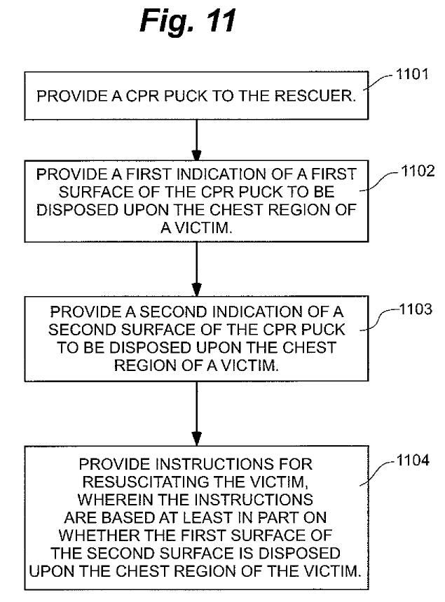

FIG. 11 illustrates generally a flow chart example of an embodiment of assisting a rescuer in attempting to resuscitate a patient using a CPR puck such as illustrated in FIG. 3 at 301. At 1101, a CPR puck is provided to the rescuer. In an embodiment, CPR puck 301 is sized and shaped to be placed on a chest region of a patient. At 1102, first indicator 303 on a first surface 304 of CPR puck 301 to be placed on the chest region of a patient is provided to the rescuer. At 1103, second indicator 315 of a second surface 314 of CPR puck 301 to be disposed upon the chest region of a patient is provided to the rescuer. At 1104, instructions are provided to the rescuer for resuscitating the patient, wherein the instructions are based at least in part on whether first surface 304 or second surface 314 is placed on the chest region of the patient.

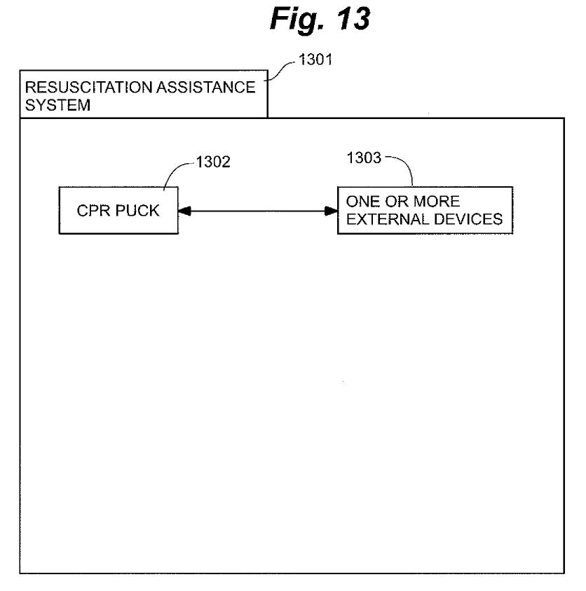

FIG. 13 illustrates generally a system for assisting a rescuer in attempting to resuscitate a patient 1301. In an embodiment, the system includes CPR puck 1302 as discussed herein. CPR puck 1302 may be communicatively coupled to one or more external devices 1303. In various embodiments, one or more external devices 1303 may be adapted to assist one or more rescuers to resuscitate a patient. In an embodiment, one or more external devices 1303 may include any of the devices discussed herein with respect to devices included in the CPR puck 1302. In one embodiment, one or more external devices 1303 may include any of the sensors discussed herein with respect to the CPR puck 1302. In various embodiments, the one or more external devices include any combination of the devices or sensors discussed herein with respect to the CPR puck 1302. In various embodiments, the one or more external devices are adapted to communicate instructions as discussed herein with respect to the CPR puck 1302.

The present invention may be embodied in other specific forms without departing from the spirit of the essential attributes thereof. Therefore, the illustrated embodiments should be considered in all respects as illustrative and not restrictive, reference being made to the appended claims rather than to the foregoing description to indicate the scope of the invention.

1. A method of assisting a rescuer to resuscitate patients, comprising:
providing a cardiopulmonary resuscitation (CPR) puck to the rescuer, wherein at least one portion of the CPR puck is sized and shaped to be disposed upon a chest region of a patient and includes at least a first surface and a second surface, the CPR puck further including:
a first indication that the first surface of the CPR puck is to be disposed upon the chest region of the patient; and
a second indication that the second surface of the CPR puck is to be disposed upon the chest region of the patient; and
providing instructions to the rescuer for resuscitating the patient that are based at least in part on whether the first surface or the second surface is disposed upon the chest region of the patient.

2. The method of claim 1, wherein providing instructions includes providing indicia on at least one of the first surface and the second surface.

3. The method of claim 1, wherein providing the CPR puck includes providing the first indication in a first language if the first surface of the CPR puck is disposed upon the chest region of the patient and providing the second indication in a second language if the second surface of the CPR puck is disposed upon the chest region of the patient; and
wherein providing instructions includes providing instructions in the first language if the first surface of the CPR puck is disposed upon the chest region of the patient and providing instructions in the second language if the second surface of the CPR puck is disposed upon the chest region of the patient.

4. The method of claim 1, wherein providing the CPR puck includes providing the first indication that indicates to the rescuer that the CPR puck is intended for resuscitating a first group of patients when the first surface of the CPR puck is disposed upon the chest region of the patient and the second indication that indicates to the rescuer that the CPR puck is intended for resuscitating a second group of patients when the second surface of the CPR puck is disposed upon the chest region of the patient.

5. The method of claim 4, wherein providing the CPR puck includes providing the CPR puck wherein the second surface of the CPR puck is adapted to generally conform to the chest region of a first group of patients, and wherein the first surface of the CPR puck is adapted to conform to the chest region of a second group of patients.

6. The method of claim 5, wherein a difference between the first group of patients and the second group of patients is selected from the set including: a difference in age, a difference in size, a difference in sex or any combination thereof; and
wherein providing the CPR puck includes providing the CPR puck such that differences between the first surface and the second surface correspond to the difference between the first group of patients and the second group of patients.

7. The method of claim 1, further comprising:
automatically recognizing whether the first surface of the CPR puck or the second surface of the CPR puck is oriented to be facing the chest region of the patient; and
wherein providing instructions is performed responsive to automatically recognizing whether the first surface of the CPR puck or the second surface of the CPR puck is oriented to be facing the chest region of the patient.

8. The method of claim 7, wherein the step of automatically recognizing whether the first surface of the CPR puck or the second surface of the CPR puck is oriented to be facing the chest region of the patient includes use of an accelerometer.

9. The method of claim 7, wherein the step of automatically recognizing whether the first surface of the CPR puck or the second surface of the CPR puck is oriented to be facing the chest region of the patient includes use of a weighted switch to determine the directions of up and down with respect to the CPR puck.

10. The method of claim 7, wherein the step of automatically recognizing whether the first surface of the CPR puck or the second surface of the CPR puck is oriented to be facing the chest region of the patient includes use of a conductive fluid in a capsule to determine the directions of up and down with respect to the CPR puck, by having the conductive fluid touch electrical contacts based on the orientation of the puck.

11. A method of a rescuer resuscitating a patient using a cardiopulmonary resuscitation (CPR) assist device, comprising:
determining, based on at least a first indication on a first surface of the CPR assist device and at least a second indication on a second surface of the CPR assist device, which one of the first surface and the second surface to place in contact with the patient;
placing the one of the first surface or the second surface of the CPR assist device in contact with a chest region of the patient; and
receiving instructions to resuscitate the patient, wherein the instructions are at least in part based on the one of the first surface or the second surface placed in contact with the chest region of the patient.

12. The method of claim 11, wherein receiving instructions includes receiving instructions selected from the set including: hand/finger placement, number of hands/fingers, chest compression rate, chest compression depth, rate of application of mouth-to-mouth resuscitation, strength of application of mouth-to-mouth resuscitation, instructions related to the use of electrical therapy, instructions related to the use of drug delivery therapy or any combination thereof.

13. The method of claim 11, wherein receiving instructions includes receiving instructions selected from the set of: receiving audio instructions, receiving visual instructions or any combination thereof.

14. The method of claim 11, wherein the at least one first indication indicates to a rescuer that the CPR assist device is intended for resuscitating a first group of patients and the at least one second indication indicates to a rescuer that the CPR assist device is intended for resuscitating a second group of patients; and
wherein determining which of the first surface and second surface of the CPR assist device to place in contact with the patient includes determining based on whether the patient is one of the first group of patients or the second group of patients.

15. The method of claim 11, wherein resuscitating includes at least one action selected from the set including: performing CarioPulmonary Resuscitation (CPR), manually initiating or adjusting electrical cardiac therapy, automatically initiating or adjusting electrical cardiac therapy, performing mouth-to-mouth resuscitation, manually delivering drugs, automatically delivering drugs or any combination thereof.

16. The method of claim 11, wherein determining which of the first surface and the second surface of the CPR assist device is in contact with the patient is performed automatically without intervention of an operator.

17. The method of claim 16, wherein a recognizer device is used to determine whether a first or second surface of the CPR assist device is in contact with the patient.

18. A resuscitation assistance device, comprising:
an object sized and shaped to be disposed upon a chest region of a patient, the object having a first surface and a second surface, wherein the object includes:
a first indication that the first surface of the object is to be placed in contact with the patient;
a second indication that the second surface of the object is to be placed in contact with the patient; and
indicia conveying instructions disposed upon the object to indicate use of the object when attempting to resuscitate the patient based upon observation of the first indication and the second indication.

19. The device of claim 18, further comprising means for automatically recognizing whether the first surface or the second surface is in contact with the patient.

20. The device of claim 19, further comprising means for automatically delivering instructions based on the means for automatically recognizing.

21. The device of claim 20, wherein the instructions include at least one action selected from the set including: hand/finger placement, number of hands/fingers, chest compression rate, chest compression depth, rate of application of mouth-to-mouth, strength of application of mouth-to-mouth, instructions related to the use of electrical therapy, instructions related to the use of drug delivery therapy or any combination thereof.

22. The device of claim 20, wherein the first indication indicates that the device will communicate in a first language and the second indication indicates that the device will communicate in a second language; and
wherein the means for automatically delivering instructions is adapted to communicate in the first language if the first surface is placed in contact with the patient to communicate in the second language if the second surface is placed in contact with the patient.

23. The device of claim 18, wherein at least one portion of the first surface is adapted to conform to a chest region of a first group of patients and at least one portion of the second surface is adapted to conform to a chest region of a second group of patients; and
wherein the first indication and the second indication indicate which of the first surface or the second surface is to be placed in contact with the patient based on whether the patient is in the first group of patients or the second group of patients.

24. The device of claim 18, wherein the object is communicatively coupled to one or more external devices, the external device being adapted assist a rescuer to attempt resuscitating a patient.

25. The device of claim 24, wherein the one or more external devices is selected from the set including: a computing device, a therapy initiation or adjustment device, a defibrillation device, a pacing device, a drug delivery therapy device, a communications device, a monitor device, a display device, a user interface, a microphone or any combination thereof.

26. The device of claim 24, wherein the at least one of the object and the at least one external device is adapted to initiate or adjust resuscitation therapy or instructions based on whether the first surface or the second surface is placed in contact with the patient.
